(19) United States
(12) Patent Application Publication
(10) Pub. No.: US 2020/0143917 A1
(43) Pub. Date: May 7, 2020

(54) SYSTEMS AND METHODS FOR MONITORING AND MANAGING CANCER PATIENTS RISK FOR ACUTE CARE UTILIZATION AND/OR FOR IMPROVING TREATMENT TOLERABILITY

(71) Applicant: NxGen Med LLC, Stoneham, MA (US)

(72) Inventor: Rakshit Sharma, Bedford, MA (US)

(73) Assignee: NxGen Med LLC, Stoneham, MA (US)

(21) Appl. No.: 16/676,381

(22) Filed: Nov. 6, 2019

Related U.S. Application Data
(60) Provisional application No. 62/756,866, filed on Nov. 7, 2018.

(51) Int. Cl. G16H 10/60; G16H 80/00; G16H 15/00; G16H 50/30; A61B 5/00

(52) U.S. Cl. CPC G16H 10/60; G16H 80/00; C12Q 1/6886; G16H 50/30; A61B 5/4848; G16H 15/00

(57) ABSTRACT

The present invention generally relates to system and methods for improving treatment tolerability for patients, for example, including remote monitoring by medical staff, tracking patient reported symptoms, and/or precision support interventions. Some aspects of the invention are generally directed to systems and methods for determining changes in a patient, and making recommendations based on such changes. For example, a variety of data representing conditions such as body weight, temperature, number of steps taken, etc. may be acquired, e.g., using a smartphone or a computer, and based on analysis of those data, one or more recommendations may be made to the patient. In some cases, the patient may also be provided with the recommended treatments, for example, a high calorie meal plan or meal supplements.

### RELATED APPLICATIONS

This application claims the benefit of U.S. Provisional Patent Application Ser. No. 62/756,866, filed Nov. 7, 2018, incorporated herein by reference in its entirety.

### FIELD

The present invention generally relates to system and methods for improving treatment tolerability for patients, for example, including remote monitoring by medical staff, tracking patient reported symptoms, and/or precision support interventions.

### BACKGROUND

One unmet need in clinical medicine and the field of oncology today is the ability to continuously monitor and remotely support the patients throughout the duration of their cancer treatments. Many signs, symptoms, and side-effects which emerge during treatment, either because of the cancer or the treatment can go unnoticed as often treatments are of long duration and patients are treated in an outpatient setting. This has huge implications on patients' ability to tolerate their treatment and eventually on the clinical outcomes and costs for both patients and for the healthcare system. Accordingly, improvements are needed.

### SUMMARY

The present invention generally relates to system and methods for improving treatment tolerability for patients, for example, including remote monitoring by medical staff, tracking patient reported symptoms, and/or precision support interventions. The subject matter of the present invention involves, in some cases, interrelated products, alternative solutions to a particular problem, and/or a plurality of different uses of one or more systems and/or articles.

In one aspect, the present invention is generally directed to a method for treating a patient having cancer, or other diseases such as those described herein. In one set of embodiments, the method comprises determining, in a patient having cancer, a numerical value associated with each of the following conditions: body weight, body temperature, physical activity, fatigue, insomnia, pain, anxiety, depression, anorexia, nausea, constipation, diarrhea, dyspnea, cognitive problems, and sensory neuropathy; determining a recommendation based on the numerical value; and treating the patient following the recommendation.

The method, in another set of embodiments, comprises determining, in a patient having cancer, a numerical value associated with each of the following conditions: fatigue, insomnia, pain, anxiety, depression, anorexia, nausea, constipation, diarrhea, dyspnea, cognitive problems, and sensory neuropathy; independently comparing each of the numerical value to a threshold numerical value; providing a recommendation if at least one of the numerical values exceeds the corresponding threshold value; and treating the patient following the recommendation.

The method, in another set of embodiments, is a method for treating a patient having cancer. In certain cases, the method comprises determining, in a patient having cancer, one or more of the following parameters: a social determinant of health, and/or a type of diagnosis, and/or a type of treatment, and/or a patient parameter, and/or a patient-reported outcome, and/or a proxy outcome; computing a risk score based on the parameters; providing a recommendation to a care provider based on the risk score; and treating the patient based on the recommendation.

In yet another set of embodiments, the method is a method for treating a patient having cancer. In some embodiments, the method comprises determining, in a patient having cancer, one or more of the following parameters: a social determinant of health, and/or a type of diagnosis, and/or a type of treatment, and/or a patient parameter, and/or a patient-reported outcome, and/or a proxy outcome; computing a risk score based on the parameters; and providing a recommendation to a care provider based on the risk score.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates one embodiment of the invention;

FIG. 2 illustrates an example of data that may be collected in accordance with another embodiment of the invention;

FIG. 3 illustrates another example of data that may be collected in still another embodiment of the invention;

FIG. 4 illustrates yet another example of data that can be collected, in another embodiment of the invention;

FIG. 5 illustrates one embodiment of the invention comprising baseline and dynamic components for risk assessment;

FIG. 6 illustrates various parameters for determining risk, in another embodiment of the invention;

FIG. 7 illustrates example recommendations base on risk, in yet another embodiment of the invention;

FIG. 8 illustrates example recommendations base on risk, in still another embodiment of the invention; and

FIGS. 9A-9B illustrate various non-limiting examples of determining patient recommendations, in another embodiment of the invention.

### DETAILED DESCRIPTION

The present invention generally relates to system and methods for improving treatment tolerability for patients, for example, including remote monitoring by medical staff, tracking patient reported symptoms, and/or precision support interventions. Some aspects of the invention are generally directed to systems and methods for determining changes in a patient, and making recommendations based on such changes. For example, a variety of data representing conditions such as body weight, temperature, number of steps taken, etc. may be acquired, e.g., using a smartphone or a computer, and based on analysis of those data, one or more recommendations may be made to the patient. In some cases, the patient may also be provided with the recommended treatments, for example, a high calorie meal plan or meal supplements.

For example, certain aspects of the present invention are generally directed to systems and methods for caring for patients having cancer, or other diseases such as diabetes or heart disease, in which monitoring of a patient's health status, such as their nutritional status, can be important in providing long-term care to the patient.

The patient may be human or a non-human mammal. In some cases, data representing one or more conditions of the subject are determined, e.g., quantitatively. These may be compared against suitable values (e.g., threshold values) to determine a suitable recommendation. In some cases, the recommendation are actually executed, i.e., the patient is treated, or provided treatment, according to the recommendation. For example, the patient may perform the treatment (for example, eating a high calorie meal plan). In some cases, multiple recommendations may be provided to the patient, e.g., based on the various conditions that are determined.

The conditions to be determined may, in some cases, be objective conditions, e.g., body weight or temperature. In some cases, the conditions may be self-reported and/or determined by a suitable care provider, e.g., fatigue, depression, etc. The conditions can in some cases be quantified, e.g., against a numerical scale. As an example, a condition such as anxiety may be quantified on an arbitrary numeric scale (e.g., 0 to 5, 1 to 5, 1 to 10, 0 to 10, 0 to 100, or 1 to 100, where either higher or lower numbers can indicate more or less anxiety, depending on the embodiment.

Non-limiting examples of objective conditions that can be determined include body weight (e.g., in kg or lb), body temperature (e.g., in Celsius or Fahrenheit), physical activity (e.g., as determined by the number of steps taken), etc. It should be understood that objective conditions such as these can each be independently determined automatically (for example, using a digital thermometer, a digital pedometer, etc.) and/or manually (for example, using an ordinary mercury or alcohol thermometer, an ordinary bathroom scale, etc.), depending on the embodiment. Manual data can then be entered, e.g., into a smartphone as discussed herein. For instance, the number of steps taken may be determined using a pedometer or using tools such as a smartphone, which can determine the number of steps taken, e.g., using accelerometers, gyroscopes, compasses, GPS positioning, etc. For instance, in some cases, the number of steps taken may be determined based on the distance actually travelled.

Non-limiting examples of conditions that may be self-reported and/or determined by a suitable care provider include fatigue, tiredness, or lack of energy (e.g., on a numeric scale of 0-5 or 1-5, where 0 or 1 is no fatigue and 5 is extreme fatigue), insomnia or sleeplessness (e.g., on a numeric scale of 0-5 or 1-5, where 0 or 1 is normal sleep and 5 is no sleep), pain (e.g., on a numeric scale of 0-5 or 1-5, where 0 or 1 is no pain and 5 is extreme pain), anxiety or worry (e.g., on a numeric scale of 0-5 or 1-5, where 0 or 1 is no anxiety and 5 is extreme worry all the time), depression or sadness (e.g., on a numeric scale of 0-5 or 1-5, where 0 or 1 is no depression and 5 is extreme sadness all the time), anorexia or appetite loss (e.g., on a numeric scale of 0-5 or 1-5, where 0 or 1 is no appetite loss and 5 is no appetite at all), nausea or vomiting (e.g., on a numeric scale of 0-5 or 1-5, where 0 or 1 is no vomiting and 5 is more than 6 episodes of vomiting in 24 hours), constipation (e.g., on a numeric scale of 0-5 or 1-5, where 0 or 1 is normal bowel movement and 5 is extreme constipation), diarrhea (e.g., on a numeric scale of 0-5 or 1-5, where 0 or 1 is no watery stools and 4 is more than 4 watery stools in 24 hours), dyspnea or shortness of breath (e.g., on a numeric scale of 0-5 or 1-5, where 0 or 1 is no shortness of breath and 4 is shortness of breath on rest), cognitive problems, forgetfulness, or problem with memory (e.g., on a numeric scale of 0-5 or 1-5, where 0 or 1 is no problem with memory and 5 is inability to remember anything), sensory neuropathy, tingling, or numbness (e.g., on a numeric scale of 0-5 or 1-5, where 0 or 1 is no tingling or numbness and 5 is extreme tingling numbness), or the like. As mentioned, the numerical scale from 0-5 or 1-5 is arbitrary and is presented here by way of example only; in other embodiments, other numeric scales (or other scales, e.g., based on faces, shapes, colors, spectra, or the like) may be used, such as the Wong-Baker scale for pain.

Such reporting may be done, e.g., manually or automatically. For example, in one embodiment, a patient may enter such data on a smartphone. In another embodiment, a care provider may determine one or more of these by asking or talking to the patient, then entering the data. As mentioned, a variety of care providers may provide assistance in reporting, such as relatives, doctors, nurses, aides, personal care aides, home health aides, nursing assistants, paralegals, nurse practitioners, or the like.

Examples of various conditions are presented here in additional detail. It should be understood that not all of these conditions may be present in a patient, and may not be determined in all embodiments; in addition, other conditions not discussed here may be determined in other embodiments. Thus, this list is intended to be by way explanation only, and should not be construed as limiting the scope of the invention to only these conditions. For example, in some cases, 2, 3, 4, 5, 6, 7, 8, 9, 10, 11, 12, 13, 14, 15, or more conditions may be determined in various embodiments, including those described below, as well as other conditions.

Body weight can be a measure of both dehydration and nutritional status. Dehydration in cancer patients is a major risk for ER visits and/or hospitalization, and may be addressed and managed upon finding. Similarly, a declining weight trend over several days could indicate either poor nutritional intake and can impact patients' abilities to tolerate their treatment and needs to be addressed. A gain in weight also needs to be investigated to rule out any edema, heart failure, or ascites during the treatment.

Body temperature may be kept track of during cancer treatment, especially to rule out febrile neutropenia, or any infections. Some patients are at high risk of hospitalization or ER visits due to high temperature.

Physical activity is a measure of functional status of the patients, and significant drops from patients' baseline levels could be early signs of distress or functional decline.

Fatigue is an important measure of a patient's quality of life, and their ability to continue with their treatment. For instance, extreme fatigue could be a sign of declining nutritional status or any underlying pathology.

Insomnia is a good measure of patient's quality of life and their ability to tolerate their treatment. Any changes from the usual could also be indicators of pain, anxiety, depression, etc.

Anxiety or worrying is a measure of a patient's quality of life and their abilities to continue with their treatment. Major changes in anxiety levels could indicate other problems.

Depression is an important component of a patient's quality of life, and prolonged or disabling depression should be investigated.

Anorexia or loss of appetite is an important symptom which, if present over a long period of time, could impact a patient's nutritional and/or functional status, and/or a patient's abilities to tolerate and continue their treatment.

Nausea or vomiting is an important symptom in patients in anticancer treatments, as it might impact their abilities to take oral drug and continue their treatments, or get severely dehydrated in case of unchecked vomiting. In addition, such patients may be at the risk of ER visits, hospitalization, or the like.

Constipation in patients may be determined, as it can be fairly common in patients undergoing cancer treatment on pain medications, or post-surgery. Constipation may also be an indicator of dehydration. In addition, insufficient fiber intake could lead to severe discomfort, impacted quality of life, and/or put the patient at the risk of hospitalization or other adverse events.

Diarrhea or loose stools is an important symptom in cancer patients, as it could be a side effect of the chemotherapy or any antibiotics which patient might have taken and needs to be addressed. Besides impacting the quality of life of the patients, diabetes comes with heightened risk of dehydration, subsequently hospitalization, or the like.

Dyspnea or shortness of breath in cancer patients could be an important symptom. It may be due to side effects of the chemotherapy, or other underlying pathologies.

Cognitive problems, forgetfulness, problems with memory, etc., may be a side effect of chemotherapy, untreated underlying depression, or the like. These could also impact a patient's abilities to continue with their treatment, or their quality of life. This may put them at risk of harm while continuing their daily activities.

Sensory neuropathy or tingling/numbness could be a side effect of chemotherapy, and if left unchecked, can be a risk for patients to continue their treatment.

As mentioned, other conditions can be determined, e.g., in addition to and/or instead of the above. For example, one condition may be the amount of lean muscle mass or lean body mass. This may be determined, for example, using a dynamometer. One non-limiting example of such an approach is described in U.S. Provisional Patent Application Ser. No. 62/750,064, filed Oct. 24, 2018, entitled “Systems and Methods for Remotely Monitoring Lean Muscle Mass,” incorporated herein by reference in its entirety.

The conditions may be determined at any rate or frequency, and the conditions may be determined simultaneously (or near-simultaneously), or at different times throughout the day. For example, a patient using an app or a computer program may be asked a series of questions to determine one or more conditions, such as the ones described herein. In some cases, the app or the computer program may also remind the patient to use other data acquisition systems, such as scales, thermometers, pedometers, dynamometers, etc. in order to generate such data.

The data may be generated at any suitable rate or frequency. For instance, the data may be obtained 1, 2, 3, 4, or more times per day, or once every 2 days, once every 3 days, once every 4 days, etc. In some cases, the data may be obtained a certain number of times per week, e.g., 1, 2, 3, 4, 5, or 6 times per week. In other embodiments, the data may be obtained at other rates, including irregular or random rates.

Thus, certain aspects of the present invention are generally directed to systems and methods for acquiring such data, and determining suitable recommendations. Optionally, this may also increase a treatment step, e.g., following the recommendations. For instance, one set of embodiments is generally directed to an app or other computer program (e.g., for a computer or a smartphone), which may optionally be placed in electronic communication with a central server or health platform. A variety of electronic communication techniques can be used, including the Internet, Bluetooth, RF communications, direct electrical connection, or the like. For example, the weight scale may transmit data directly, via cellular signal, via Bluetooth connectivity, etc. to the app and/or the health platform. Similarly, the thermometer may transmit data either directly, cellular signal, via Bluetooth connectivity, etc., to the app and/or to the health platform. Those of ordinary skill in the art will be aware of apps, computer programs, and suitable programming techniques. Such programs can be run on any suitable device, e.g., on computers, smartphones, dedicated devices, or the like.

Optionally, the systems and methods can include other sources of data, such as scales, thermometers, pedometers, dynamometers, or the like, e.g., as discussed herein. Such data sources may be in electrical communication with the app or other computer program, and/or with the central server or health platform. The central server or health platform may process the data (which may come from one or more sources, as discussed) to produce one or more suitable recommendations, e.g., using a suitable recommendation engine. An example of an engine that is able to generate recommendations is discussed in detail herein, although it should be understood that the engine, including techniques for determining suitable recommendations, is by way of example only, and that in other embodiments, other recommendation engines may be used, e.g., applying different numerical values, providing different recommendations, acquiring data representing different conditions instead of and/or in addition to the ones discussed herein.

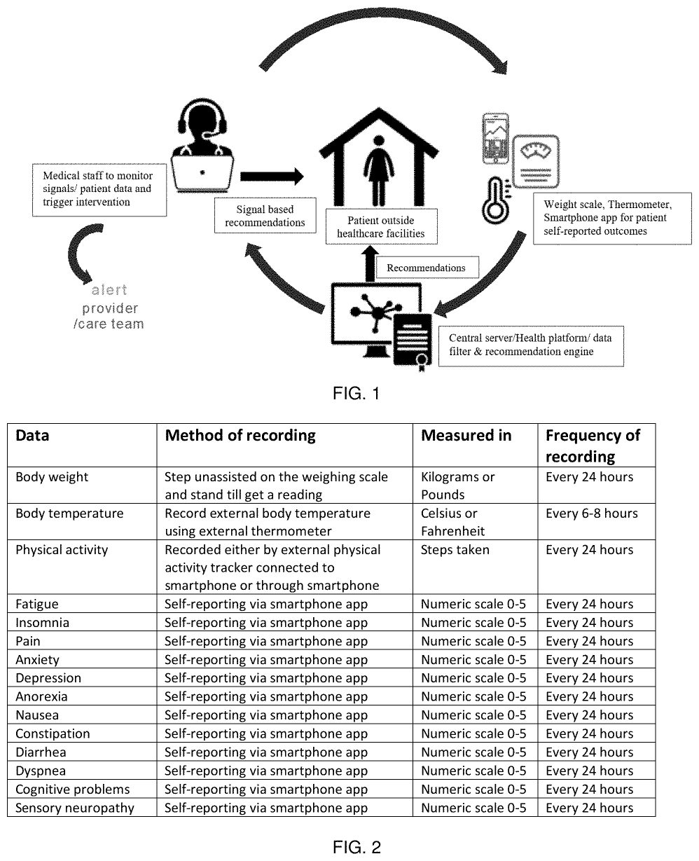

Thus, as a non-limiting example, an embodiment of the invention includes a weighing scale, a thermometer, an app (or other computer program), and a central server/health platform. In some cases, the weighing scale and/or the thermometer may be in electronic communication with the app and/or with the central server or health platform. The app may be running on, e.g., a computer, a smartphone, a dedicated device, or the like. Non-limiting examples of smartphones include the iPhone or an Android phone. In one embodiment, the central server/health platform/recommendation engine may be designed to detect changes along with readings and filter signals across different data streams, e.g., either in time and/or in absolute changes, and depending upon the magnitude of change, can either offer recommendations or trigger alert to care team/medical staff for further investigation. A non-limiting example of such an implementation is shown in FIG. 1.

As a specific non-limiting example, a patient may use a connected weighing scale (e.g., one that is Bluetooth or cellular enabled), a connected thermometer (e.g., one that is Bluetooth or cellular enabled), and a link to smartphone app (e.g., iPhone, Android, etc.). The patient may weigh themselves every 24 hours on the weighing scale, check their body temperature every 6 hours, and open the smartphone app once every day to report their progress. An example of the data that may be collecting using this system is shown in FIG. 2. Thus, for example, the weight scale may transmit the body weight (e.g., in kg or lb) to the health platform (and/or to the app). Similarly, the thermometer may transmit body temperature (e.g., in Celsius or Fahrenheit) to the health platform (and/or to the app), and the app may transmit patient self-report numerical score for the symptoms to the health platform. The app also, in some embodiments, can receive alerts, reminders, messages, recommendations or communications from the central server/health platform, e.g., depending upon the patient score, recommendations to be provided to the patient, or the like.

In some embodiments, one or more recommendations may be provided to the patient, and in some cases, the recommendations may actually be administered to the patient, e.g., by a suitable care provider, by self-administration by the patient, etc. For example, a recommendation to a patient may include the patient eating a high calorie meal plan or a meal supplement, taking anti-fever medication, increasing exercise (e.g., increase average daily steps by 10%), yoga, relaxation techniques, medical evaluation, or the like. In addition, in certain embodiments, the recommendations may include adjustments to the drug dosing of one or more drugs being given to the patient. For example, a reporting score of 4 or 5 (out of 5) may result in a recommendation that a drug dose be decreased by 5%, 10%, 25%, 50%, or even that a drug be removed. Other examples of recommendations include any of those described herein.

In some cases, such recommendations may be triggered upon a condition achieving a certain value, for example, a self-reported value or an objective condition such as those described herein. As a non-limiting example, if the self-reported values can range between 0 and 5, a numerical value of 0 or 1 may not require any action, while a numerical value of 2 or 3 may require minor interventions (for example, one of the ones discussed in this paragraph), while a numerical value of 4 or 5 may require medical intervention and suitable treatment). In other embodiments, however, other scales (e.g., with numeric ranges, pictures, etc.), may be used.

Another aspect is generally directed to assessing, monitoring, and/or managing patients, e.g., that are potentially at risk for acute care utilization. For example, various conditions of the patient may be determined, and compared to one or more respective thresholds. In some cases, one or more respective recommendations may be provided, and optionally used to treat the subject. The subject may have cancer or other diseases such as those described herein.

For example, acute care utilization (defined as an unplanned patient visit to the emergency department, unplanned hospitalization or unplanned readmission post hospitalization discharge) in cancer patients undergoing active treatment (chemotherapy, surgery, radiation therapy, hormone therapy, immunotherapy) is often a major problem that can compromise clinical outcomes and/or add significantly to the overall cost of cancer care. Many oncology treatments have moved to an outpatient basis, and there are no effective means to monitor patients throughout their cancer treatment. For instance, such patients may not be supported at home in between their medical visits.

In some embodiments, one or more parameters may be determined for a patient, e.g., one that has cancer. These may include, for example, one or more of a social determinant of health (e.g., age, income, education, gender, family support, food insecurity, transportation, etc.); a type of diagnosis (e.g., a site of cancer, a type of cancer, tumor staging, any preexisting comorbidities, etc.); a type of treatment (e.g., chemotherapy, surgery, radiation therapy, immunotherapy, hormone therapy, etc.); a patient vital or other parameter (e.g., based of the type of cancer, any or all of patient's daily weight, daily temperature, daily physical activity, blood pressure, blood glucose, blood oxygen saturation, heart rate, etc.); a patient reported outcome (e.g., fatigue, sleep, anxiety, sadness, appetite loss, nausea, constipation, diarrhea, shortness of breath, memory problem, numbness, etc.); and/or a proxy reported outcome (e.g., one that is collected by medical staff remotely, such as any information patient supplies to care professional remotely on their condition or symptoms during routine check in with the patient).

In some cases, a risk scoring system may be used. The risk scoring system may be qualitative (e.g., high, medium, low risk), and/or quantitative (e.g., a numerical score). Risk assessment, in certain embodiments, may be performed as a dynamic assessment, and/or in real-time. The risk, in some cases, may be a risk of acute care utilization, and may be updated (e.g., on a daily basis) during the patient's treatment. Our risk scoring system comprises of multiple inputs/parameters, including a social determinant of health, a type of diagnosis, a type of treatment, a patient parameter, a patient-reported outcome, and/or a proxy outcome. Non-limiting examples of these include those given above. In some cases, some of these may be baseline and/or dynamic components, and may be used to determine risk stratification, e.g., as a risk score. One non-limiting example of such a system is provided in FIG. 5.

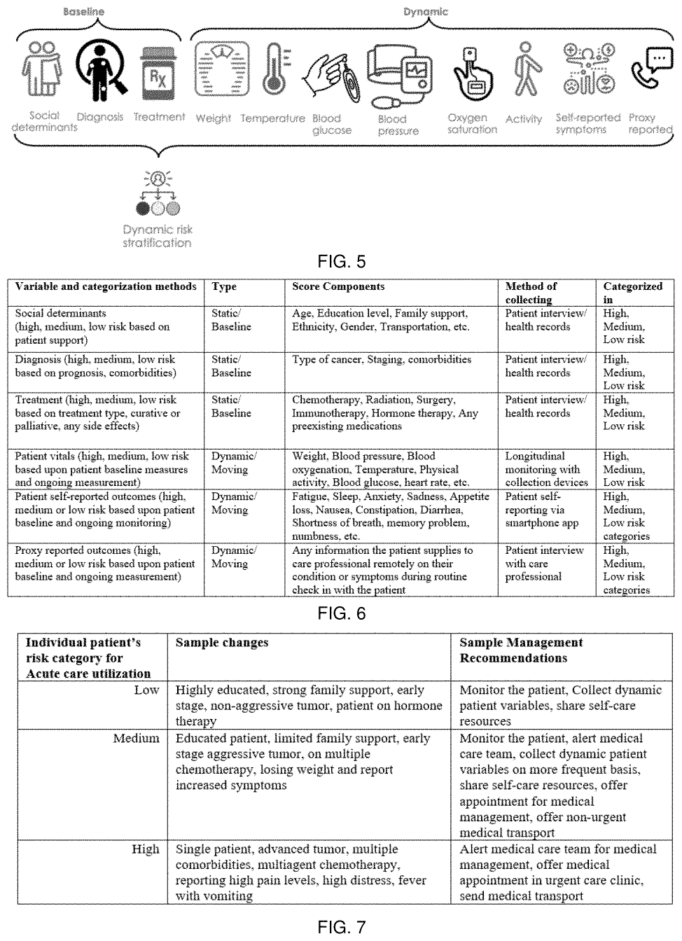

In some cases, the patient risk stratification or risk score may be computer. For example, based upon the multiple input criteria (both fixed/static like social determinants of health, type of diagnosis, type of treatment and dynamic/changing variables like patients vitals, patient reported outcomes and proxy reported outcomes which are collected on a regular basis), patients may be stratified into high, medium and low risk categories of acute care utilization. The risk score may be calculated (e.g., continuously or in real-time, or on a daily basis, etc.), as biometric data is updated. Examples of biometric data include daily weight, blood pressure, temperature, patient self-reported outcome measures like sleep, anxiety, pain, etc., or other parameters such as any of those described herein. A non-limiting example of such a system is shown in FIG. 6.

In some embodiments, such risk scoring or stratification may be used to provide a recommendation, e.g., to the patient or a care provider, such as relatives, doctors, nurses, aides, personal care aides, home health aides, nursing assistants, paralegals, nurse practitioners, or the like. The recommendation may be based on the risk score. The professional can then opt to treat the patient, e.g., based on the recommendation, or take other appropriate actions.

A patient's risk scoring or stratification can be utilized in a variety of ways. For example, at an individual patient level, such as can be risk scoring or stratification can be provided to a care provider, e.g., transmitted for visualization and/or communication to the care provider (for instance, through a web-portal, through Electronic Medical Records system, etc.). In some cases, this may be used as an early warning or alerting system for appropriate management of patient and patient's risk for acute care utilization. A non-limiting example of such a utilization scheme is shown in FIG. 7.

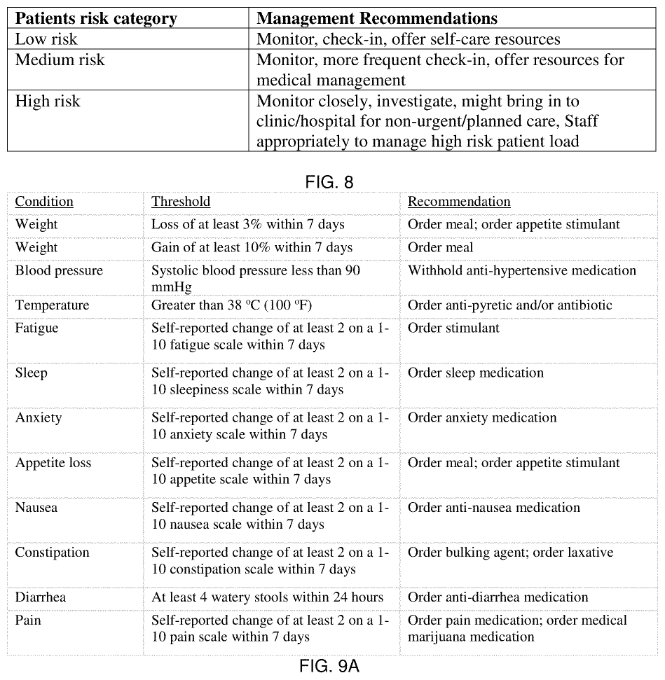

As another example, at a pooled population level, a plurality of cancer patients being monitored at individual clinic, hospital, health system, health network, health plan, population health, etc. may be pooled/stratified according to their risk category for visualization and/or communication to care providers for appropriate risk management (for instance, through a web-portal, through Electronic Medical Records system, etc.). A non-limiting example of such a utilization scheme is shown in FIG. 8.

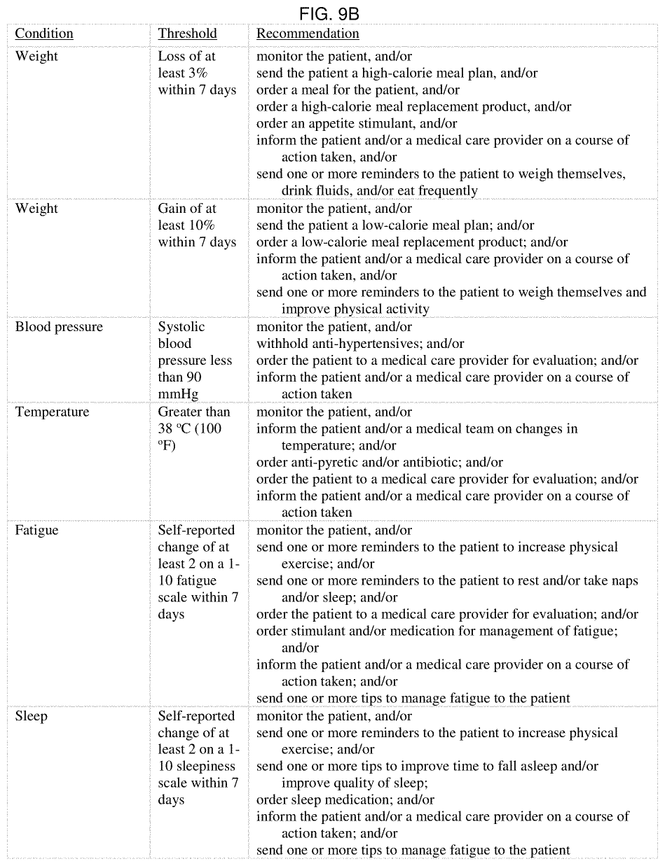

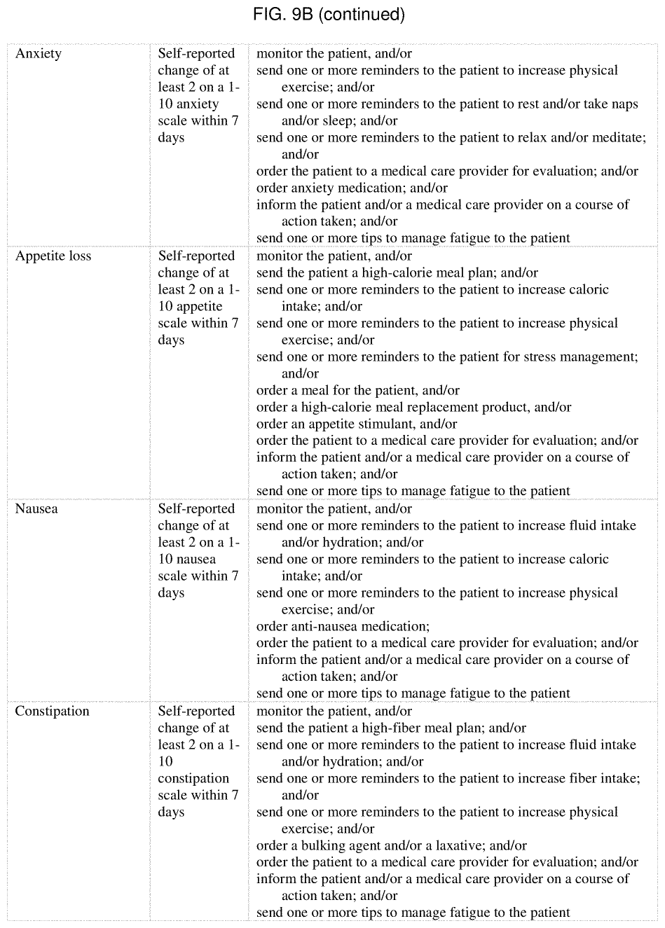

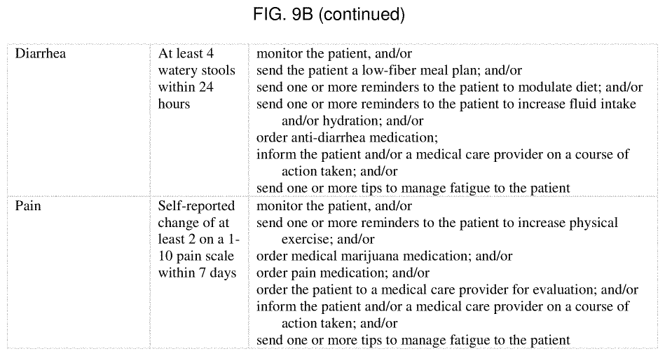

Non-limiting examples of a risk management system in accordance with certain embodiments of the invention are now described. The system may be able to monitor cancer patients and their dynamic data streams to analyze changes in patient vitals, patient self-reported outcomes and proxy reported outcomes to automates a host of management options with a managed oversight as patient risk condition changes during the course of treatment. These examples are shown in FIGS. 9A and 9B, and may include any one or more of the listed recommendations.

It should be understood that conditions, thresholds, and recommendations are provided herein solely by way of example only, and not limitation. For example, any 1, 2, 3, 4, 5, or more of the conditions may be determined, i.e., not every condition may be determined in every embodiment. Similarly, one of ordinary skill in the art may use thresholds or metrics other than those described herein. For instance, scales (such as appetite scales, fatigue scales, sleepiness scales, anxiety scales, nausea scales, constipation scales, pain scales, etc.) may operate on different ranges (e.g., 1-5, 1-20, 1-100, etc.), and/or the time reported for change may be determined over a different number of days (e.g., 1, 2, 3, 4, 5, 6, 7, 8, 9, 10, 11, 12, 13, 14, 15, etc.) before a threshold change is reached. Such scales may be determined, e.g., as averages or moving averages over the number of days. Similarly, the threshold change (e.g., at least 1, at least 2, at least 3, at least 4, at least 5, at least 7, at least 10, at least 20, etc.) may be different in different embodiments, e.g., depending on the scale used.

It should be understood that in certain embodiments, such changes are relative. For instance, a first patient may report a regular (baseline) score of 3 on a scale, and a threshold change of at least 2 means that a recommendation may be provided and/or action taken based on such recommendations when the score (over a certain number of days) goes below 1 or above 5. A second patient, however, may report a regular (baseline) score of 6 on such a scale, so the threshold values would be 4 and 8. Thus, a score of 6 might prompt action for the first patient, but not for the second patient.

Other changes may be determined besides the ones shown in FIGS. 9A and 9B. For example, for weight, the loss may be at least 1%, at least 2%, at least 3%, at least 4%, at least 5%, at least 6%, at least 8%, at least 10%, etc., and/or a gain of at least 1%, at least 2%, at least 3%, at least 4%, at least 5%, at least 6%, at least 8%, at least 10%, at least 12%, at least 15%, etc., over a certain number of days (e.g., 1, 2, 3, 4, 5, 6, 7, 8, 9, 10, 11, 12, 13, 14, or 15 or more days, etc.). As another example, the temperature may be greater than 39° C., greater than 40° C., greater than 41° C., greater than 42° C., greater than or 43° C., etc. As yet another example, for blood pressure, the systolic blood pressure may be less than 100 mmHg, less than 95 mmHg, less than 90 mmHg, less than 85 mmHg, less than 80 mmHg, less than 75 mmHg, less than 70 mmHg, less than 65 mmHg, less than 70 mmHg, etc., and/or the diastolic blood pressure may be at least 120 mmHg, at least 125 mmHg, at least 130 mmHg, at least 135 mmHg, at least 140 mmHg, at least 145 mmHg, at least 150 mmHg, at least 155 mmHg, at least 160 mmHg, etc.

In some cases, the patient may use a smartphone app. As a non-limiting example, the medical system may personalize and/or automates graded interventions based upon patient risk category, ongoing patient dynamic risk assessment and monitoring for optimal patient outcomes. For instance, if a patient in a risk category reports a high anxiety level, which is unusual for their baseline, the system can trigger an automated message to the patient to see if they would like to get appointment for a psychiatrist. In contrast, if a patient in a low risk category reports high anxiety levels, the system can trigger an option if patient would like to see guided meditation audio/videos.

Or, as another non-limiting example, if a low risk patient reports higher than normal pain or nausea levels for their baseline, the patient smartphone app can trigger a message if they would like to have medication ordered or delivered. In contrast, if a high risk patient reports higher than normal pain or nausea, the system may trigger a message if the patient would like to schedule an urgent care or doctor visit, and then based on the individual patient response, order medication delivery, or schedule an urgent care or doctor appointment with a preferred provider, and/or schedule a non-urgent medical transport and simultaneously alert their care provider or medical team of the actions taken.

U.S. Provisional Patent Application Ser. No. 62/750,064, filed Oct. 24, 2018, entitled “Systems and Methods for Remotely Monitoring Lean Muscle Mass,” is incorporated herein by reference in its entirety. In addition, U.S. Provisional Patent Application Ser. No. 62/756,866, filed Nov. 7, 2018, entitled “Systems and Methods for Improving Treatment Tolerability for Patients,” is also incorporated herein by reference in its entirety

The following examples are intended to illustrate certain embodiments of the present invention, but do not exemplify the full scope of the invention.

### Example 1

This example illustrates one embodiment of the present invention. However, it should be understood that the conditions described in this example, and the numerical values presented, are by way of example only, and that in other embodiments, other conditions in addition to or instead of these, and/or different numerical values for one or more of these, may be used instead.

In this example, a patient (for example, one having cancer) is provided with an app, e.g., on a smartphone or a computer, upon which the patient (and/or a suitable care provider) is able to enter data relative to various conditions experienced by the patient. The data may be entered, e.g., numerically, graphically, etc., and may be entered at various frequencies (e.g., once per day, twice per day, once every other day, etc.). In some cases, additional objective conditions may also be measured, e.g., body weight, body temperature, physical activity, etc. For example, the patient may have a scale or a thermometer, which may measure weight or temperature, respectively, and provide such data to the app. Physical activity can be measured, for example, using a pedometer (which may provide such data to the app), or in some cases, the app may be part of a smartphone that can measure physical activity.

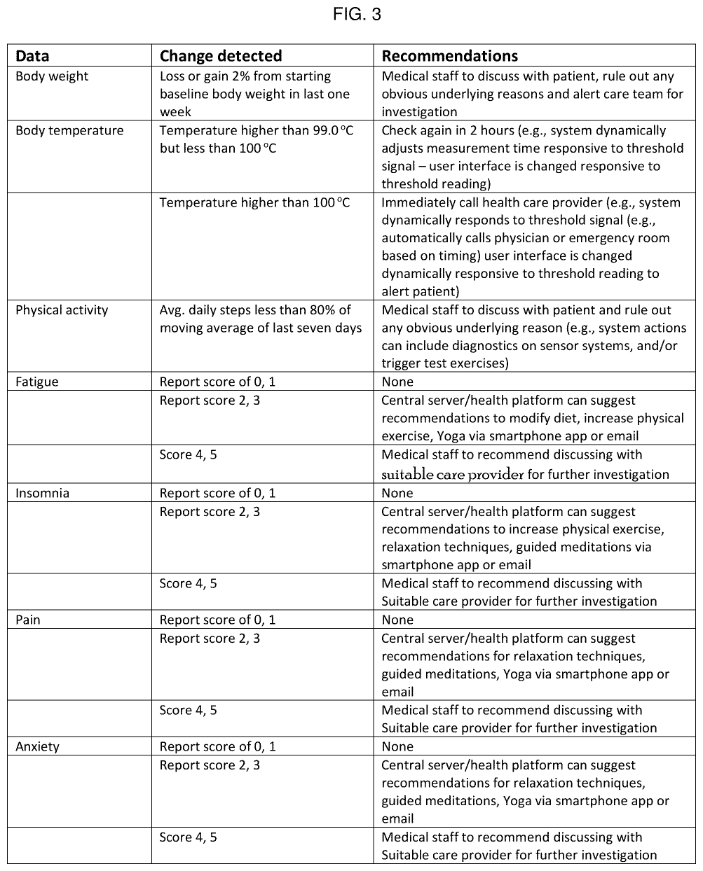

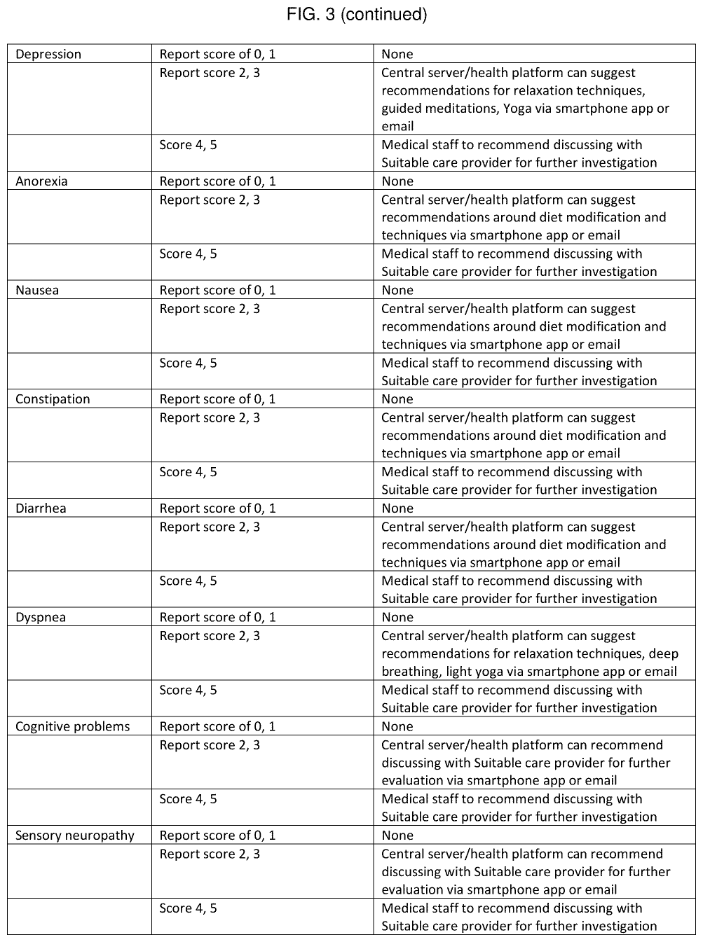

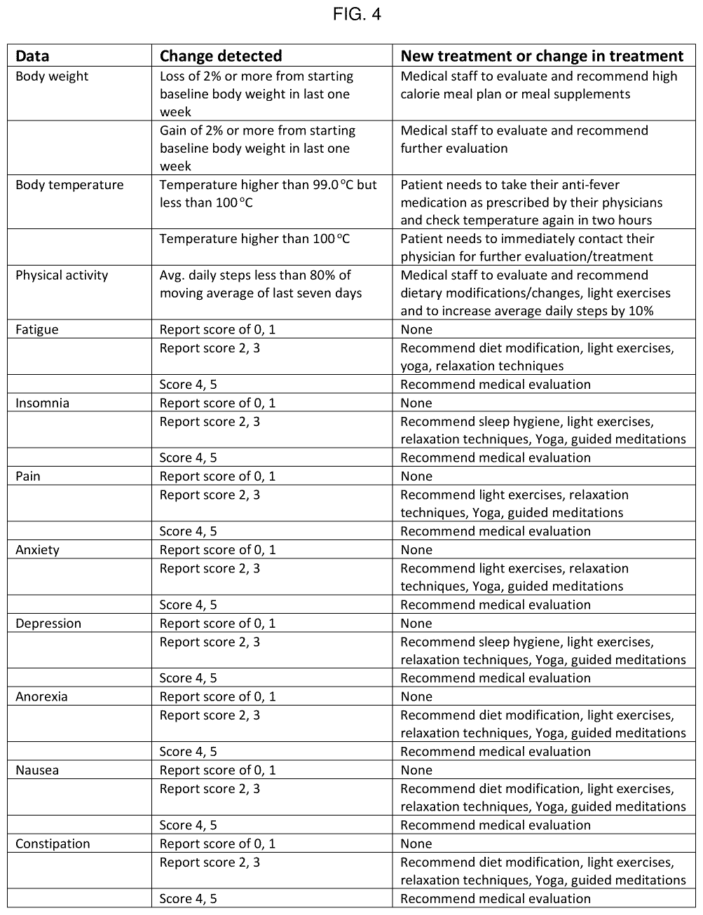

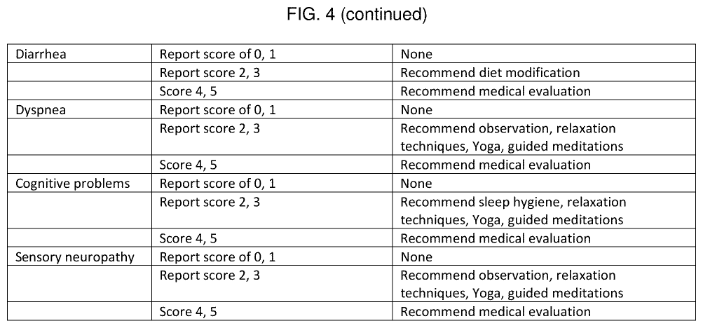

Based on these data, the app may determine suitable recommendations, e.g., in consultation with a central server or a heath platform. In some cases, the app may transmit information to medical staff, which may monitor such data and determine when to intervene, e.g., with suitable treatment, as is shown in FIG. 3. In some cases, the measurement or analysis may be used to provide new treatments and/or changes in treatment. Examples of this are shown in FIG. 4.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. (canceled)

2. A method for treating a patient having cancer, the method comprising:
determining, in a patient having cancer, each of the following conditions, using the corresponding method:

| | |
|---|---|
| Body weight | Determine with a scale |
| Body temperature | Determine with a thermometer |
| Physical activity | Determine using a pedometer or a smartphone |
| | able to measure physical activity tracker |
| Fatigue | Self-reporting on a scale of 0-5 |
| Insomnia | Self-reporting on a scale of 0-5 |
| Pain | Self-reporting on a scale of 0-5 |
| Anxiety | Self-reporting on a scale of 0-5 |
| Depression | Self-reporting on a scale of 0-5 |
| Anorexia | Self-reporting on a scale of 0-5 |
| Nausea | Self-reporting on a scale of 0-5 |
| Constipation | Self-reporting on a scale of 0-5 |
| Diarrhea | Self-reporting on a scale of 0-5 |
| Dyspnea | Self-reporting on a scale of 0-5 |
| Cognitive problems | Self-reporting on a scale of 0-5 |
| Sensory neuropathy | Self-reporting on a scale of 0-5 |

independently comparing each of these to a threshold value as follows, and supplying a corresponding recommendation:

| | | |
|---|---|---|
| Body weight | Loss or gain 2% from | Provide high calorie meal |
| | baseline body weight in | plan and/or meal |
| | last week | supplement |
| Body | Temperature higher than | Investigate with medical |
| temperature | 99° C. but less than | staff |
| | 100° C. | |
| | Temperature higher than | Take anti-fever medication |
| | 100° C. | |
| Physical | Average daily steps less | Modify diet and/or increase |
| activity | than 80% of moving | exercise and/or increase |
| | average of last seven | average daily steps by 10% |
| | days | |
| Fatigue | Report score of 0, 1 | None |
| | Report score 2, 3 | Modify diet and/or increase |
| | | exercise and/or yoga and/or |
| | | apply relaxation techniques |
| | Score 4, 5 | Investigate with medical |
| | | staff |
| Insomnia | Report score of 0, 1 | None |
| | Report score 2, 3 | Increase sleep and/or |
| | | increase physical exercise |
| | | and/or perform yoga and/or |
| | | apply relaxation techniques |
| | | and/or apply meditation |
| | Score 4, 5 | Investigate with medical |
| | | staff |
| Pain | Report score of 0, 1 | None |
| | Report score 2, 3 | Increase physical exercise |
| | | and/or perform yoga and/or |
| | | apply relaxation techniques |
| | | and/or apply meditation |
| | Score 4, 5 | Investigate with medical |
| | | staff |
| Anxiety | Report score of 0, 1 | None |
| | Report score 2, 3 | Increase physical exercise |
| | | and/or perform yoga and/or |
| | | apply relaxation techniques |
| | | and/or apply meditation |
| | Score 4, 5 | Investigate with medical |
| | | staff |
| Depression | Report score of 0, 1 | None |
| | Report score 2, 3 | Increase sleep and/or |
| | | increase physical exercise |
| | | and/or perform yoga and/or |
| | | apply relaxation techniques |
| | | and/or apply meditation |
| | Score 4, 5 | Investigate with medical |
| | | staff |
| Anorexia | Report score of 0, 1 | None |
| | Report score 2, 3 | Modify diet and/or increase |
| | | physical exercise and/or |
| | | perform yoga and/or apply |
| | | relaxation techniques and/or |
| | | apply meditation |
| | Score 4, 5 | Investigate with medical |
| | | staff |
| Nausea | Report score of 0, 1 | None |
| | Report score 2, 3 | Modify diet and/or increase |
| | | physical exercise and/or |
| | | perform yoga and/or apply |
| | | relaxation techniques and/or |
| | | apply meditation |
| | Score 4, 5 | Investigate with medical |
| | | staff |
| Constipation | Report score of 0, 1 | None |
| | Report score 2, 3 | Modify diet and/or increase |
| | | physical exercise and/or |
| | | perform yoga and/or apply |
| | | relaxation techniques and/or |
| | | apply meditation |
| | Score 4, 5 | Investigate with medical |
| | | staff |
| Diarrhea | Report score of 0, 1 | None |
| | Report score 2, 3 | Modify diet |
| | Score 4, 5 | Investigate with medical |
| | | staff |
| Dyspnea | Report score of 0, 1 | None |
| | Report score 2, 3 | Observe subject and/or |
| | | perform yoga and/or apply |
| | | relaxation techniques and/or |
| | | apply meditation |
| | Score 4, 5 | Investigate with medical |
| | | staff |
| Cognitive | Report score of 0, 1 | None |
| problems | Report score 2, 3 | Increase sleep and/or |
| | | perform yoga and/or apply |
| | | relaxation techniques and/or |
| | | apply meditation |
| | Score 4, 5 | Investigate with medical |
| | | staff |
| Sensory | Report score of 0, 1 | None |
| neuropathy | Report score 2, 3 | Observe subject and/or |
| | | perform yoga and/or apply |
| | | relaxation techniques and/or |
| | | apply meditation |
| | Score 4, 5 | Investigate with medical |
| | | staff |

and
treating the patient following the recommendation.

3-5. (canceled)

6. A method for treating a patient having cancer, the method comprising:
determining, in a patient having cancer, one or more of the following conditions;
comparing the one or more conditions to a respective threshold;
providing one or more respective recommendations; and
treating the patient following the recommendation.

| Condition | Threshold | Recommendation |
|---|---|---|
| Weight | Loss of at least 3% within 7 days | Order meal; order appetite |
| | | stimulant |
| Weight | Gain of at least 10% within 7 days | Order meal |
| Blood pressure | Systolic blood pressure less than 90 | Withhold anti-hypertensive |
| | mmHg | medication |
| Temperature | Greater than 38° C. (100° F.) | Order anti-pyretic and/or antibiotic |
| Fatigue | Self-reported change of at least 2 on a 1- | Order stimulant |
| | 10 fatigue scale within 7 days | |
| Sleep | Self-reported change of at least 2 on a 1- | Order sleep medication |
| | 10 sleepiness scale within 7 days | |
| Anxiety | Self-reported change of at least 2 on a 1- | Order anxiety medication |
| | 10 anxiety scale within 7 days | |
| Appetite loss | Self-reported change of at least 2 on a 1- | Order meal; order appetite |
| | 10 appetite scale within 7 days | stimulant |
| Nausea | Self-reported change of at least 2 on a 1- | Order anti-nausea medication |
| | 10 nausea scale within 7 days | |
| Constipation | Self-reported change of at least 2 on a 1- | Order bulking agent; order laxative |
| | 10 constipation scale within 7 days | |
| Diarrhea | At least 4 watery stools within 24 hours | Order anti-diarrhea medication |
| Pain | Self-reported change of at least 2 on a 1- | Order pain medication; order |
| | 10 pain scale within 7 days | medical marijuana medication |

7. The method of claim 6, further comprising monitoring the patient.

8. The method of claim 6, further comprising informing a medical care provider of the status of the patient.

9. The method of claim 6, further comprising monitoring the patient.

10. The method of claim 6, further comprising transporting the patient to a medical care provider.

11-12. (canceled)

13. A method for treating a patient having cancer, the method comprising:
determining, in a patient having cancer, one or more of the following parameters:
a social determinant of health, and/or
a type of diagnosis, and/or
a type of treatment, and/or
a patient parameter, and/or
a patient-reported outcome, and/or
a proxy outcome;
computing a risk score based on the parameters;
providing a recommendation to a care provider based on the risk score; and
treating the patient based on the recommendation.

14. The method of claim 13, wherein the risk score is numerical.

15. The method of claim 13, wherein the risk score is high risk, medium risk, or low risk.

16. The method of claim 15, wherein if the risk score is low risk, the recommendation comprises:
monitor the patient; and/or
send one or more reminders to the patient.

17. The method of claim 15, wherein if the risk score is medium risk, the recommendation comprises:
monitor the patient; and/or
send one or more reminders to the patient; and/or
offer resources to the patient.

18. The method of claim 15, wherein if the risk score is high risk, the recommendation comprises:
monitor the patient; and/or
send one or more reminders to the patient; and/or
order the patient to a medical care provider for evaluation.

19. The method of claim 13, wherein the social determinant comprises age, income, education, gender, family support, food insecurity, and/or transportation.

20. The method of claim 13, wherein the type of diagnosis comprises a site of cancer, a type of cancer, tumor staging, and/or a comorbidity

21. The method of claim 13, wherein the type of treatment comprises chemotherapy, surgery, radiation therapy, immunotherapy, and/or hormone therapy.

22. The method of claim 13, wherein the patient parameter comprises the daily weight of the patient, the daily temperature of the patient, the daily physical activity of the patient, the blood pressure of the patient, the blood glucose of the patient, the blood oxygen saturation of the patient, and/or the heart rate of the patient.

23. The method of claim 13, wherein the patient-reported outcome comprises fatigue, sleep, anxiety, sadness, appetite loss, nausea, constipation, diarrhea, shortness of breath, memory problem, and/or numbness.

24. (canceled)
